(19) United States
(12) Patent Application Publication
(10) Pub. No.: US 2024/0252389 A1
(43) Pub. Date: Aug. 1, 2024

(54) WEARABLE AND PORTABLE SMART ACTUATION DEVICE FOR DVT RISK MITIGATION: DEEP VEIN THROMBOSIS PREVENTION DEVICE (DVT-PD)

(71) Applicant: Drexel University, Philadelphia, PA (US)

(72) Inventors: Hassan El Mghari, Philadelphia, PA (US); Gregory Olsen, Landsdale, PA (US); Zikang Ling, Philadelphia, PA (US); Srivatsa Rayar Ganesh, Garnet Valley, PA (US); William Mongan, Coatesville, PA (US); Vasil Pano, Portland, OR (US); Kapil R. Dandekar, Philadelphia, PA (US)

(73) Assignee: Drexel University, Philadelphia, PA (US)

(21) Appl. No.: 18/560,487

(22) PCT Filed: May 16, 2022
(86) PCT No.: PCT/US2022/029408

§ 371 Date: Nov. 13, 2023

Related U.S. Application Data
(60) Provisional application No. 63/188,678, filed on May 14, 2021.

(51) Int. Cl. A61H 15/00; A61H 9/00; A61N 1/04; A61N 1/36; G16H 40/63

(52) U.S. Cl. CPC A61H 15/0078; A61H 9/0078; A61N 1/0452; A61N 1/36031; G16H 40/63; A61H 2201/10; A61H 2201/123; A61H 2201/164; A61H 2201/165; A61H 2201/1654; A61H 2201/501; A61H 2201/5084; A61H 2209/00; A61H 2230/085

(57) ABSTRACT

A deep vein thrombosis prevention device (DVT-PD) is a wearable device for the lower extremities senses the user's dynamic or static movements and actuates accordingly to lower DVT risks. The device uses a comprehensive control system together with an integrated machine learning model. to classify, manage, direct, and regulate signals and the behavior of the device.

### STATEMENT REGARDING GOVERNMENT SUPPORT

This invention was made with government support under Contract No. NIH-U01EB023035 awarded by the National Institute of Health. The government has certain rights in the invention.

### BACKGROUND

### 1 Background

### 1.1 Introduction

Deep vein thrombosis (DVT) is a serious medical condition that affects up to 900,000 people annually and 1 in 20 people. This occurs when a blood clot forms in a vein, leaving individuals susceptible to short-term complications such as pulmonary embolism. Currently, DVT poses a higher threat due to the global pandemic. COVID-19 has exacerbated the risks of DVT for hospitalized patients, many of whom are immobilized after operation. DVT is a serious medical condition that occurs when a person's blood coagulates and eventually forms into a blood clot in veins that are located deep in the body. A blood clot, also known as thrombus, occurs when red blood cells group together and convert from a liquid into a solid state and stop the blood from flowing through the veins and back to the heart. DVT mainly affects the large veins in the lower leg and thigh and a visual representation is shown in FIG. 1.

These blood clots can form anywhere in the body but are usually prevalent in the thigh and/or lower leg. The human body's blood circulation and organ functionality rely on the blood's ability to move freely throughout the closed loop network, made up of arteries and veins. It is especially dangerous when a blood clot forms and breaks free to flow back to the lungs and ends up getting trapped in the lungs' pulmonary artery, resulting in pulmonary embolism (PE). The condition that occurs when a person develops DVT and subsequently PE is called VTE, as shown in FIG. 2.

DVT can also cause a variety of other harmful health conditions such as postphlebitic syndrome, swelling in foot, ankle, or leg (typically, on one side), cramping in leg or calf, unexpected pain in foot and ankle, having an area of skin that feels relatively warmer than areas surrounding it and/or skin turning red or blue in color. For the upper extremities (a blood clot in the arm), DVT common symptoms include neck, shoulder, and arm pain, swelling, change in color, and weakness in hands are other symptoms as well.

Current devices on the market such as Graduate Compression Stockings (GCS), Intermittent Pneumatic Compression (IPC), and Venous Foot Pumps (VFP) help promote circulation through means of compression. Although these preventative devices are designed to alleviate the risks of developing DVT, there are concerns regarding effectiveness and patient comfort.

### 1.2 Motivation and Problem Description

The motivation of the DVT-PD project is to help lower the number of people who are at risk of developing Deep Vein Thrombosis (DVT), specifically patients undergoing ambulatory surgeries, and to ultimately save lives. Roughly 300,000-900,000 people develop DVT in the United States every year, with an alarmingly high mortality rate of 10-30%. DVT is the U.S.'s leading cause of all maternal deaths, meaning death occurred during or shortly after pregnancy or delivery. Every year, 48 million ambulatory surgeries put patients who were previously at a high risk of developing DVT, at an even greater risk during these operations due to immobility and their inability to be on anticoagulants.

Moreover, COVID-19 has put many lives in jeopardy and has led to an increase in the number of bedside patients in hospitals. Recent studies performed in America and Russia show high incidence of Venous Thromboembolism (VTE) in patients with moderate to severe COVID-19, indicating that these patients may require an early administration of anticoagulation therapy, as well as mechanical prophylaxis treatment of the lower extremities.

There are multiple risk factors that contribute to the development of DVT in the body and some of them include smoking, birth control, malignancy, obesity, genetics, chronic lower extremity lymphedema, prolonged bed rest, injury or surgery, cancer, heart failure, inflammatory bowel disease, old age, and sitting for long periods of time (driving or plane rides). The most serious risk factors are long periods of immobilization, pregnancy, and obesity. The risk factor that DVT-PD is designed to directly combat is immobilization. Another significant risk factor for DVT is Chronic Venous Insufficiency (CVI), a condition that occurs when the walls and/or valves located in the veins in the lower leg are negatively compromised and do not work properly. CVI ultimately decreases the amount of blood returning to the heart from the legs and can increase the chance of developing a blood clot.

There are various forms of treatment for the prevention of DVT and they can vary for each individual patient. The most popular commercially available treatment is anticoagulants, also known as blood thinners. The other type of treatment that is primarily used in the hospital setting designed for post-operation patients are DVT preventative devices. These preventative DVT medical devices include Intermittent Pneumatic Compression (IPC) devices, compression stockings, Venous Foot Pumps (VFP), and may be discussed in greater detail below.

One of the most effective ways to prevent DVT is staying physically active, which maintains the circulation of blood in the deep veins of one's lower extremities, thereby preventing the formation of a blood clot during an extended period of stasis. However, long periods of physical inactivity occur frequently for people at risk of DVT, and these periods of inactivity have increased during the global pandemic. The goal for many prospective DVT patients becoming more physically active is not realistic, hence the importance of mechanical prophylaxis methods for DVT prevention; especially for those who find maintaining compliance with anticoagulant medication difficult.

The ultimate goal of almost all DVT preventative solutions, whether mechanical or chemical, is to increase the circulation of the blood in the deep veins in the lower extremities during long periods of physical inactivity and/or recovery.

### 1.3 Literature, Market, and Existing Solutions Survey

As briefly mentioned above, due to the prevalence and severity of DVT, many physical solutions were devised to combat it. The Graduated Compression Stockings (GCS), as shown in FIG. 3 on the left, are the most common and have the advantage of being more acceptable since they are portable. The stockings exert varying amounts of compression to different parts of the leg, with the greatest pressure at the ankle and lowest pressure at the top of the stockings. The pressure gradient ensures that the blood of the extremity flows upwards towards the heart, while increasing the blood flow velocity and volume. However, GCS have many contraindications and a 30%-65% noncompliance rate. They are also much less effective than their mechanical prophylaxis counterparts.

The Intermittent Pneumatic Compression (IPC), on the other hand, applies a sequential compression, mostly with inflation of air, and prevents DVT formation through two mechanisms: By decreasing venous stasis and activating fibrinolysis, which is the enzymatic breakdown of fibrin in blood clots. The IPC, shown in FIG. 3 middle, is the most common mechanical prophylaxis method used in a surgical setting for patients.

The Venous Foot Pump (VFP) inflates a small balloon that compresses the bottom of the foot, which activates the plantar venous plexus and sends blood to the heart. The VFP in FIG. 3 right mimics the action of walking and increases blood circulation that way.

As mechanical prophylaxis solutions for DVT, the IPC and VFP have similar limitations.

They both confine the patient to a bed and prohibit the patient from walking. This is a significant disadvantage of the devices that lead to low compliance rates of approximately 60%.24 In addition, the VFP needs to be fitted professionally to the patient, which adds another challenge, and has a rate of 59% patients who are not fitted correctly.

### SUMMARY OF THE EMBODIMENTS

### 1.4. Summary

DVT-PD strives to fill in the gap for what the existing solutions cannot offer and provide a better and more effective experience for the user. DVT-PD may absorb the advantage of the GCS, in term of mobility, and the advantages of the IPC and the VFP, in terms of effective prevention of DVT and elevation of blood flow velocity, while perfecting upon them to deliver an improved product. Table 1, FIG. 25 compares the ease of use of DVT-PD with devices discussed above.

### 1.4.1 The Need and Problem Statement

In order to achieve the goal of effectively preventing DVT and even VTE, improving lower limb circulation and reducing blood stasis are important. The device and system described herein alleviates and prevents risks of DVT development by increasing both venous and arterial blood flow and reducing blood stasis in the lower extremities.

The wearable device, for the lower extremities, senses the user's dynamic or static movements and actuates accordingly to lower DVT risks. The design uses a comprehensive control system, together with an integrated machine learning model, to classify, manage, direct, and regulate signals and the behavior of the device.

FIG. 4 shows a hardware and software block diagram developed for DVT-PD to. DVT-PD may include several blocks: Sensing, microcontroller, and actuation hardware.

These blocks work together to enable DVT-PD to work effectively and may be described herein in more detail.

### 1.4.2 Target Specifications

Some target specifications of DVT-PD may include:
The target increase of mean blood flow velocity in deep veins of the leg
The target compression pressure while wearing the device
The actuation compression pressure gradient (for the compression actuation method)
The minimum runtime from a single charge
The weight of the device

The first target specification is increasing the mean blood flow velocity in the deep veins of the legs by 150% from the baseline mean blood flow velocity before actuation. A potential margin of error of −25% is anticipated and accepted from a targeted 150% increase. As far as an upper limit for blood flow velocity, a dramatic increase in the mean blood flow velocity in deep veins may not be an area of concern, even for healthy adults. If a patient's resting blood flow velocity is measured to be 20 cm/s at rest, then it may be increased to 50 cm/s after actuation (or 45 cm/s as the minimum). The most common product that hospitals and doctors use to prevent DVT from developing before, during, and after ambulatory surgeries is IPC and it was found that IPC is able to increase the patient's blood flow velocity by roughly 117.3% in the deep veins in the thigh. DVT-PD is being designed with the goal of improving upon former devices used in mitigating DVT, hence the 150% mean blood flow velocity goal. The method and tool responsible for evaluating the user's mean blood flow velocity may be done by using a venous ultrasonic doppler monitoring device (See below).

The second target specification is the baseline pressure applied by merely wearing the DVT-PD. This is crucial to the operation of DVT-PD without adding risk or medical complications to the user. According to doctors in the field, a device with a wearable pressure greater than 12 mmHg puts the patient in risk of blood clots by cutting off the capillary veins in the leg. This information is why the maximum wearable pressure without actuation was chosen to be 10 mmHg, with a marginal error of +2 mmHg.

The third target specification is the compressive pressure gradient required to increase the user's deep vein mean blood flow velocity. It should be noted that this specification is relevant in the case of the compression actuation method. A pressure gradient of 20 mmHg applied to the lower leg was proven to be effective in supplying the right pressure to combat blood pooling and stasis. Compression stockings have been shown to decrease the risk of developing DVT with pressures ranging from 15 mmHg to greater than 49 mmHg, therefore a pressure gradient of 20 mmHg is sufficient. The target pressure gradient may also be allowed to vary from 20 mmHg by +3 mmHg to stay in the proven acceptable compression pressure range.

The fourth target specification is the minimum runtime that DVT-PD can perform from a single charge. In order for DVT-PD to be portable, it may need to be powered by a rechargeable battery (potentially with an amplification driving circuit). DVT-PD's ideal target runtime may be at least 4 hours with a marginal error of +1 hour. This runtime was selected after analyzing the average operation and post-operation ambulatory surgery times. According to the National Hospital Ambulatory Medical Care Survey (NHAMCS), it was found that 48.3 million ambulatory surgeries were performed in the US in 2010, and the average time in operating room was 57 minutes, with an average post-operation time of 70 minutes. The DVT-PD may treat all ambulatory surgeries, a possible runtime of 4 hours was chosen so that it can easily operate during the longer surgeries and provide pre- or post-op care as well.

The last target specification requires DVT-PD to be a total weight of approximately 3 pounds with a marginal error of +1 pound. Since DVT-PD may be wrapped around a large portion of the lower leg as a wearable device, the total weight of 3 pounds may be distributed over a portion the lower leg, minimizing weight felt by the user. This target weight was chosen with the goal of having DVT-PD suitable for people of all ages, athletic abilities, and strengths. The target specifications mentioned above are shown in FIG. 25, Table 2.

The codes and standards adhered to for this project, from a manufacturing standpoint, are shown below in FIG. 27, Table 3.

### BRIEF DESCRIPTION OF THE DRAWINGS

FIG. 1 shows examples of deep vein thrombosis in a leg vein.

FIG. 2 shows a venous thromboembolism (VTE).

FIG. 3 shows graduated compression stockings, intermittent pneumatic compression, and a venous foot pump.

FIG. 4 shows a DVT-PD hardware and software block diagram.

FIG. 5 shows an integrated system of a DVT-PD.

FIG. 6 shows an EMG sensor.

FIG. 7 shows a triple-axis accelerometer.

FIG. 8 shows a block diagram of the machine learning model.

FIG. 9 shows a working principle of DEAs.

FIG. 10 shows an NMES overview.

FIG. 11 shows an EMS Digital Neuromuscular NMES stimulator.

FIG. 12 shows a waveform of a tested NMES stimulator mode and electrode replacement.

FIG. 13 shows a DVT-PD control design.

FIG. 14 shows an overview of the DVT-PD.

FIG. 15 shows an ultrasonic Doppler monitor display image.

FIG. 16 shows shows a hardware block diagram of the DVT-PD.

FIG. 17 shows a data collection schematic.

FIG. 18 shows a data collection wiring schematic.

FIG. 19 shows the data collection prototype.

FIG. 20 shows a simulated date collection.

FIG. 21 shows a DC-DC boost converter circuit.

FIG. 22 shows an integrated timing circuit.

FIGS. 23 and 24 show oscilloscope wave output.

FIG. 25 shows Table 1.

FIG. 25 shows Table 2.

FIG. 27 shows Table 3.

FIG. 28 shows Table 4.

FIG. 29 shows Table 5.

FIG. 30 shows Table 6.

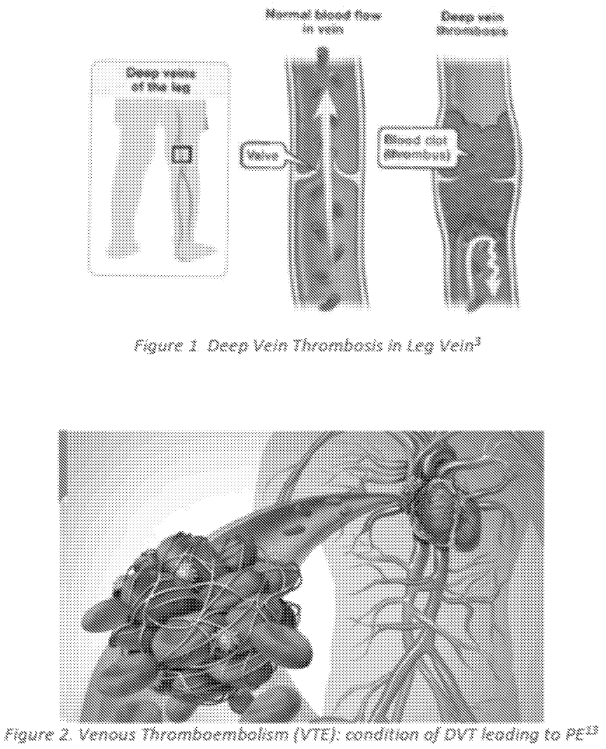

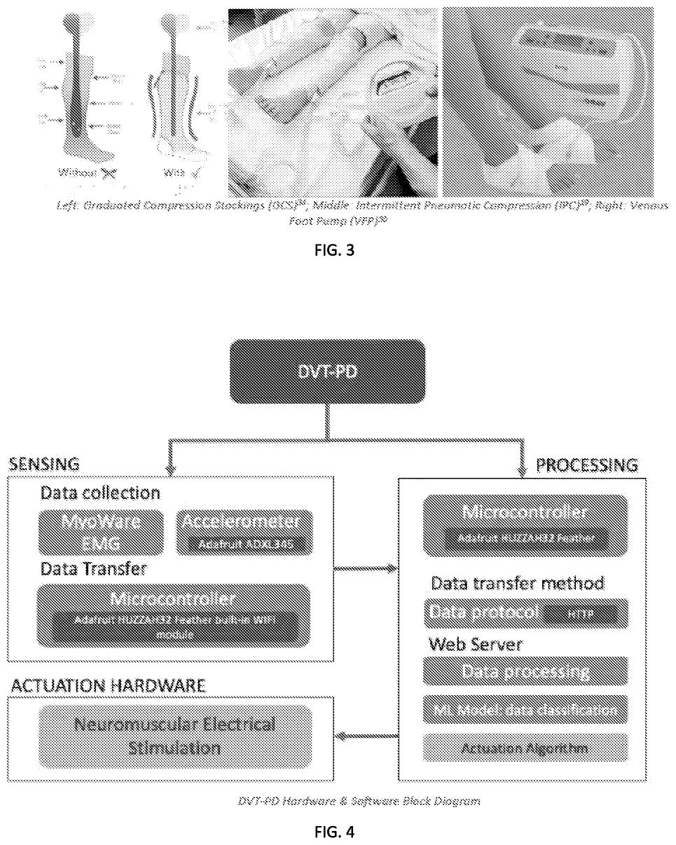

### DETAILED DESCRIPTION OF THE EMBODIMENTS

2 Design Description

This section describes the sensing, machine learning, actuation method, and control system—in more detail including evaluation of concepts to aid in the team's decision-making process.

### 2.1 Concepts

The DVT-PD uses a comprehensive control system that works with a machine learning model to sense the user's dynamic or static movements. Upon receiving data from the sensor, the machine learning model may classify, manage, direct, and regulate signals and, together with the control system, command the device to actuate when appropriate. The DVT-PD may include the following goals:
Device does not immobilize the user
Device has sensing mechanisms that observes risk within the user, both dynamically and statically
Device actuates when risky behavior is observed
Device has an integrated machine learning model for classifying activity state
Device is portable, lightweight, comfortable, and unobtrusive to the user

### 2.2 Detail Design

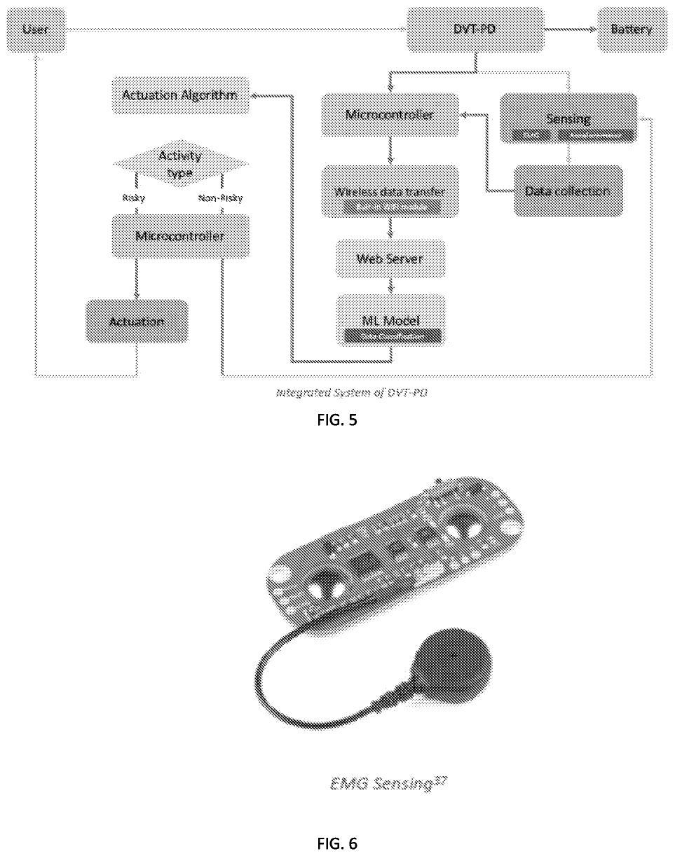

FIG. 5 shows a comprehensive system that may be integrated into DVT-PD, which is a detailed expansion of the software and hardware diagram presented in FIG. 4. Each component of the system is discussed in the subsequent section.

### 2.2.1 Sensing

### 2.2.1.1 EMG

Electromyography, also referred to as EMG, may be selected as a sensing method because it is a direct way to measure muscle activity (see diagram in FIG. 4). Sensing can be done intramuscularly, where needles are inserted into the muscle to measure the potential difference, or with a less invasive approach with pads applied to the skin. Because a minimally invasive design that was easily replicable and safe worked well in in the trial performed with the DVT-PD beta version, a surface mounted EMG was used despite them perhaps being less accurate. A surface mounted EMG may also allow for easy setup and maintenance due to the single-use pads. For the EMG sensor, the MyoWare muscle sensor was chosen due to the adjustable gain and wide input voltage range. This device is shown in FIG. 6.

### 2.2.1.2 Accelerometer

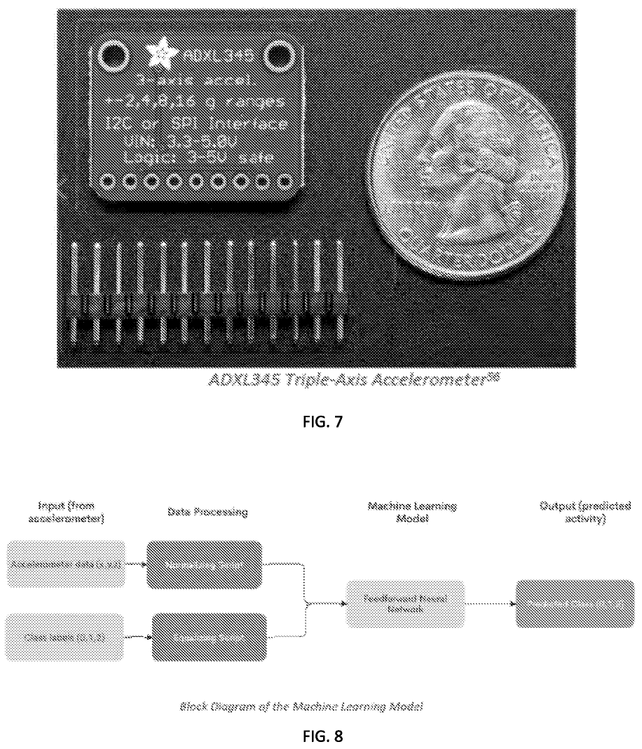

An accelerometer may be the second portion of the sensing system (as seen in FIG. 4). Moreover, it has been shown to be effective in activity recognition. It may measure the motion of the leg, which indirectly measures muscle activity. This may result in the data being triple axis spatial coordinates for the x, y, and z planes. The accelerometer that was selected for the beta testing was the ADXL345 triple-axis accelerometer, specifically chosen for its small form factor, cheap price, and ability to be weaved into a wearable device. This ensured it may be easy to setup and may be easily replicable. This device is shown in FIG. 7.

### 2.2.1.3 Machine Learning

The sensing system may be the input to the machine learning model, which comprises of a classifier. A classifier may be built to take in accelerometer data in each time frame and assign it to a class. The three main parts of classifier design may be sensing, feature selection, and classifier design. Sensing was already addressed in the past two subsections.

Wn represents the window size, which may be 6.5 seconds. For the accelerometer, the features that are proposed are the average value of the accelerometer data, the root mean squared value, and the variance.

wn=[μan,rms(an),var(an)] (2)

Three classifier designs may be considered. A long-term short-term memory recurrent neural network, a convolutional neural network, and a single layer feedforward network. The recurrent neural network was not expected to perform well because it takes previous data into consideration while the current use-case does not care what a subject was doing X minutes ago, what they are doing at the point of measurement. Convolutional neural networks are mostly used with imaging data, but a 6-layer convolutional neural network was created and evaluated by the inventors anyway to see how it may impact the data.

To evaluate the models, the runtime and accuracy were 2 factors considered. In terms of the runtime, all models performed similarly so greater emphasis was placed on the accuracy of a model. After testing all the different models on synthetic data, the single layer feed forward network performed the best in terms of accuracy and was chosen as the current model of choice for the classifier. This could change after more meaningful features are incorporated into the classifier design, and another test of all 3 models may be run once again at that point. This may take in new accelerometer data and output whatever class is most probable given the training data. A block diagram of how the machine learning algorithm works is shown in FIG. 8. Input data from the accelerometer is fed into two scripts that equalize the number of elements in each class and normalize the values. This then gets fed into the machine learning model which outputs the predicted classes for that time period.

As discussed, the classifier may take in new accelerometer data and may output whatever class it thinks more closely models the data. Another part of the system may deal with whether to actuate. Other considerations include potentially actuating on a fixed schedule (I.e., every 30 minutes), allowing patients to adjust the level of actuation, and gamifying actuation based on how many steps a patient needs to take, prescribed by a doctor.

The proposed architecture (FIG. 6) may collect the data from the accelerometer, sending it to a flask web server via a PUT request, and doing the machine learning processing on the server. The classifier may be deployed to the same web server. The classifier and the flask web server may be continuously running on an Ubuntu machine. After the classifier outputs classes over certain time periods—10 minutes at a time for example—this may be fed into the part of the system that deals with whether to actuate, which may also live in the server and was discussed in the previous paragraph. Periodically, the server may communicate with the microprocessor to decide on whether to actuate and for how long.

### 2.2.2 Actuation

Some considerations when operating the DVT-PD may involve figuring out how long, how frequently, and when to actuate. The DVT-PD may be designed to be as safe as possible for patients while also being significantly more effective than the current products available in hospitals. The patients are at the highest risk of developing a blood clot or for their blood to start pooling while they are sedated. As such, patients may receive constant actuation while under sedation. This may force DVT-PD to have 2 main modes of operation: One that is constantly actuating (forced) while ignoring the sensing data, and one where DVT-PD's decision of actuating is “smart” by relying on sensing the patients' activity.

While the patient is in surgery, the device may be in the forced constant actuation mode since that patient is unconscious and at the highest risk. DVT-PD may be in this constant actuation mode until the patient has woken up from sedation. Immediately after sedation, the device may be switched to the sensing actuation mode. This may require the medical provider to find the appropriate intensity of the electrical signal that is applied to the patient's lower legs. A pamphlet, with instructions on how to find the best intensity for each patient, may be provided to the medical provider in order to have the highest rate of compliance and effectiveness. The pamphlet may also be provided so that the patient has a better understanding of how DVT-PD works and how to use it properly and safely.

While the device is in sensing mode, DVT-PD may be classifying numerous patient activities as delineated in FIG. 28, Table 4, but not all activities may require actuation. There are two classes of activity where DVT-PD may not actuate due to patient safety concerns; these classes are when the patient is walking and when the patient is standing. DVT-PD may not actuate during these two classes because patients could fall due to their muscles contracting. Inside the patient's pamphlet may be several bed-side exercises that may be recommended to keep their blood flow up and minimize the risk of developing. DVT-PD may also not actuate if it is sensing that these exercises are being done on a regular basis or if the patient has been walking/active. The frequency of the actuation depends on if the patient is at low, medium, or high risk of developing DVT. More research is needed before the team finalizes the frequency of actuation once inactivity has been detected.

Deciding DVT-PD's actuation method is one of the considerations in the DVT-PD. The inventors explored many methods and analyzed them based on the objectives listed in herein. Of all eight actuation methods explored, three stood out and were considered to meet the target specifications while solving the problem statement. Three stood out:
a. Dielectric Elastomer Actuator (DEA) uses a smart material to actuate through electrical-signal-controlled compression
b. Neuromuscular Electrical Stimulation (NMES) applies an electrical signal to stimulate and mimic the natural muscle contraction when walking
c. Motor Actuated Compression (MAC) employs a DC motor to regulate compression

The three methods are thoroughly discussed in sections below, while Table 4 shows some pros and cons of each method.

### 2.2.2.1 Dielectric Elastomer Actuator (DEA)

Although compression to the lower part of the body helps blood return and can be crucial in the prevention of DVT, current solutions such as IPCs and VFPs may not be suitable for ambulatory use. Dielectric Elastomer Actuators (DEAs) were introduced recently as active compression bandages (ACBs) that address the shortcomings of the previous devices. Due to its special material properties, DEAs can actuate in the forms of a time-dependent mechanical compression, which was shown to be more efficient than a static compression. Controlled by an input electrical stimulus, DEAs are lightweight, portable and very fast with experimental responses as short as 2 ms.

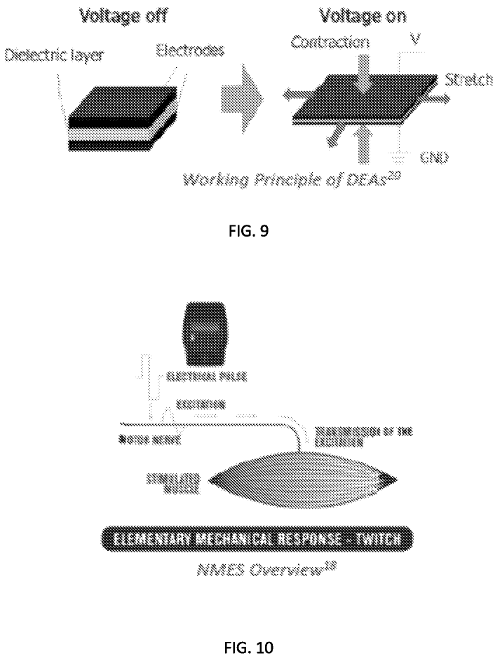

DEAs belong in the family of electro-active polymers (EAPs), which are smart polymers that changes in size or shape when stimulated by an electric field. A simple DEA unit includes a dielectric elastomer that is sandwiched between a pair electrodes. When voltage is applied to the DEA electrodes, the electrostatic Maxwell pressure leads to the expansion of the dielectric elastomer as shown in FIG. 9.

### 2.2.2.2 Neuromuscular Electrical Stimulation (NMES)

Neuromuscular electrical stimulation (NMES) is a common method used by medical professionals where electrical stimulation is applied to the patient's motor nerves as shown in FIG. 10. The goal of neuromuscular stimulation is to visibly contract the calf muscles in order to mimic the calf pump action. This calf pumping action is responsible for having the venous blood from the deep veins in the lower calf return to the heart, which is crucial for preventing blood pooling in the lower extremities.

NMES is used for aiding muscle recovery, increasing blood flow, and mimicking the brain's signal that is sent to the muscle when calling for muscle contraction or movement. NMES has two main components: the waveform generator and the electrodes. The electrical signals used for NMES treatment, which has higher frequencies between 20-50 Hz when compared to other types, can be altered to the patient's comfort and optimal outcome. The electrical signal is sent through the two electrodes with one electrode being the cathode and the other the anode. Therefore, the cathode may be at the lower calf motor point or muscle belly since the current flows from the cathode through the anode.

The NMES signals may be sent in pulses with a pulse width time around a few-hundred microseconds and can have different signal ramp-up and ramp-down times, usually 0.5-1 second. It was found that with a pulse width of 350 us, frequency of 36 Hz, 500 ms ramp-up time, 1 s ON time, 500 ms ramp-down time, expelled blood flow was maximized while maintaining patient comfort”.

### 2.2.2.3 Motor Actuated Compression

Motor actuated compression may also improve blood circulation and reduce risks of coagulation. Specific types of motors that could be used in this application include DC motors, which are commonly used as actuators because they produce continuous movement and can be controlled by their speed of rotation easily. Particularly, brushed DC motors may be ideal in this case, which may include rotational and stationary parts called rotors and stators respectively. These are motors that produce a magnetic field in the rotor by running an electrical current through a brush assembly. Moreover, the motion of the DC monitor can be converted into a linear motion that is compressive through the use of an electric linear actuator.

### 2.2.3 Evaluation of Actuation Methods

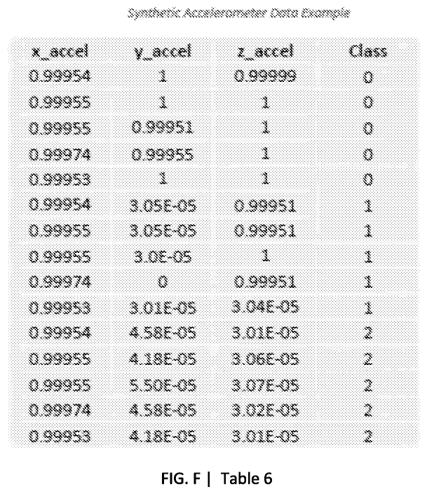

FIG. 29, Table 5 explores certain advantages and disadvantages of actuation methods.

DEA has shown that it can meet the required compression pressure of 20 mmHg. Though the input voltage signal has some control of the pressure variation, stacking up at least 20 layers of the DEA actuation units provides significant increase in actuation pressure. Additional layers may not drive up the cost to build of DVT-PD too much since dielectric material is relatively inexpensive and can be found commercially. However, the fabrication process demands high precision and involves applying a shadow mask and carbon crease coating to prevent the formation of short circuit across the circuit. This process is highly time intensive and inefficient that can be done manually.

The higher the input voltage signal being sent to DEA, the more strain and compression generated, which adds flexibility to the actuation compression pressure generated. However, the driving voltage signal needed to create the desired strain and compression in DEA is between 1-3 kV, which is extremely high for a wearable device. The high power consumption may add challenges, such as safely insulating the DEA material, creating a circuit that can amplify a battery's voltage up to 1-3 kV, all while still meeting the target runtime on a single charge lasting 4 hours. For an active compression application, the actuator needs to be constantly charged and discharged during compression, which reduces the lifetime due to constant electrical strain. DVT-PD is intended to be reused many times without the need of repair or replacement of the product, therefore the shorter lifecycle of DEA is concerning.

There are several important pros and cons of NMES to consider when analyzing if it can meet all of DVT-PD's target specifications. NMES is superior at increasing the mean blood flow velocity in the deep veins in the lower body. Studies comparing the effectiveness of increasing mean blood flow velocity between IPC and NMES show that NMES can be over three times as effective. IPC increased the users' mean blood flow velocity by 117.3%, whereas the NMES was shown to increase their mean blood flow velocity by 399.8% when the intensity of NMES was high enough for visible muscle activity (contractions). At a lower intensity, providing more comfort to the user, NMES still outperformed the IPC by achieving an increase in velocity by 150.6%. The effectiveness that NMES displayed at a lower intensity is encouraging since NMES's biggest flaw is possible patient discomfort. This suggests that NMES's intensity can be altered to substantially increase the mean blood flow velocity, all the while optimizing the patient's comfort, and ultimately patient compliance. NMES has shown to have a high rate of compliance when compared with other traditional methods and devices used to combat the development of DVT. Another flaw of NMES is its anticipated acceptability among maternal patients, who might not feel comfortable with electrical signals on their bodies in fear of potential impact of fetal health. Due to the nature of its operation, NMES also faces restrictions with patient conditions, such as patients with pacemakers and other possible heart conditions.

MAC is reliable method of actuation, similar to previously discussed options because DC brushed motors are easily controlled, small and cheap. Size plays a role in this application, as the motor should not cause any discomfort for the user when the calf is compressed. Some of the major highlights for this actuation approach are delineated in Table 4. Assembly of a DC motor is relatively simple and cheap because it requires basic parts that are accessible: Axle, rotors, stators, commutators, field magnets, and brushes. This makes material costs favorable. In addition, there is more flexibility in terms of controlling the pressure applied, which should be determined based on disparate patient risk factors and conditions. Similarly, to NMES, MAC requires a low voltage supply to operate, which is safer and correlates with a lower overall cost.

Conversely, there are other factors that may suggest another method is better. One disadvantage, which is up to the discretion of the user, is the noise radiated from the motors that can create discomfort. When the direction of electricity is switched, there may be occasional sparks that contribute to the electrical noise that is heard. MAC also may require higher maintenance and as such, may causes issues and interfere with the objective to increase patient compliance. Finally, there is a possibility the force might be distributed unevenly. In situations where compression is focused in a specific area of the lower extremity, this can be detrimental to the user of the device.

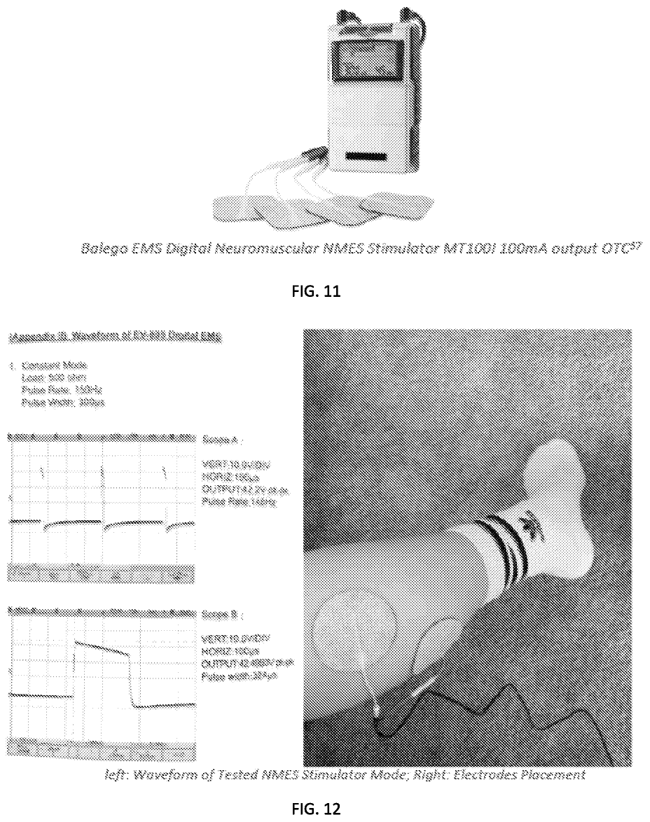

After careful evaluations and additional stakeholder interviews, neuromuscular stimulation was the chosen winner due to its portability, size, and effectiveness in increasing the blood flow as mentioned earlier. However, in order to thoroughly investigate the feasibility and efficacy of the method, the inventors prepared a trial to justify this decision. A commercialized NMES stimulator, the Balego EMS Digital NMES stimulator MT100I, (FIG. 11) was purchased and tested for this purpose.

One of the team members tested the Balego NMES stimulator on the Constant Mode (scope B), of which the waveform is shown in FIG. 12 left, with the electrodes placed on the upper gastrocnemius muscle (FIG. 12 right). This is a superficial two-headed calf muscle located on the back and the member was able to achieve constant muscle contraction without feeling physical irritation.

With neuromuscular stimulation as the option to promote muscle contraction, one thing to consider is the associated waveform that is designed to target specific areas of the muscle and provide customized forms of energy transfer. Different waveforms correspond to variances in energy transfer which can help with certain situations that are based on the patients' respective conditions. For DVT-PD's application, a biphasic waveform was tested and shown to be effective; this is a two-phase bidirectional wave—either symmetrical or asymmetrical—that is prevalent in battery powered NMES devices. Biphasic current works well with most devices that feature control settings of intensity, voltage, current, and duration of pulses. Furthermore, it can be used to strengthen the patient's muscle and increase circulation to reduce the chances of blood clots and DVT.

To provide a more cost-effective and simplistic option, the inventors also present a stimulation device. DVT-PD may feature 2 main NMES components as previously mentioned: A waveform generator and a pair of electrodes. Like the setup of the Balego commercial stimulator, the electrodes may be placed on each lower leg of the patient, where electrical signals are sent out of the anode, pass through the skin, fat, and muscle tissue, and then travel through the cathode.

The basic setup on the breadboard may feature an opto-isolator, which separates the high and low voltage sides to prevent the circuit from frying. The opto-isolator may also limit the amount of current that goes through, a safety factor to be considered when current is transferred to the patient. In addition, the setup may include a dc-to-dc voltage amplifier and a potentiometer to adjust the voltage that may be transmitted to the muscle to promote contraction. Having a comprehensive system and design that is equivalent to the actions of a commercially available stimulation device may prove beneficial in the long-run due to its lower cost and size.

Currently, designing and testing of the NMES circuit is being explored. The goal of the NMES circuit output is to produce a periodic square pulse waveform with a pulse width of 300-400 us, a frequency of 10-50 Hz, and current of 20-100 mA. The two main components that may comprise the NMES circuit are a DC-DC boost converter and an integrated circuit timer to achieve the desired waveform. Results from the circuit design are discussed later in more detail.

### 2.2.4 Decision Matrix

The decision matrix is an important used for this project to systematically identify and analyze the best candidate. For this case, a decision matrix was created to solidify a decision based on the 3 actuation methods considered: DEA, NMES, and MAC. Several categories that pertain to each option were considered and weighed accordingly based on their significance and contribution. Specifically, emphasis was placed on safety, performance, and business side of the methods. It is important to ensure the choices are minimally invasive, best serve the stakeholder group established, and are beneficial to the user. Once the respective weights of each members were put in, simple algebraic manipulation was used to tally the scores and determine the best choice. Once the scores were tallied, it was determined that NMES was the best option to pursue (Appendix C).

### 2.2.5 Control System

Part of designing any smart device is establishing a control system that may organize the logic that may be applied during the use of DVT-PD. The initial control system design is broken up into three main components of sensing, classifying, and actuating. This preliminary setup is illustrated below in FIG. 14. The first part of the control system is for DVT-PD to be sensing the user's movements and/or muscle activity (depending on what sensing methods are the most effective). This data may then be sent to the machine learning model which may classify the patient's activity and movements as previously mentioned in Section 2.2.2. After DVT-PD classifies the patient's movement, it may determine if the patient is either at risk or not at risk of developing DVT. If the ML classifier determines that the patient has been active enough and is not at risk of blood pooling, then DVT-PD may loop back and continue to keep sensing the patient's movements. However, if the classifier determines that the patient has been inactive for a specified amount of time, DVT-PD may warn the user twice that actuation may be taking place (either sound, vibration, or both), and then actuate accordingly. The warnings may take place two minutes before actuation to give the patient and/or medical provider enough time to prepare for actuation (if they are walking or standing). The warnings are chosen to be both vibration and auditory in case the patient lacks in one of the two senses, and the auditory signal gives a chance for the medical providers or other surrounding people to be aware as well. The vibration and auditory signals may be coming from DVT-PD device. The team considered having the reminders connected to an app on the patient's smartphone, but that may be applicable if the patient owns a smartphone and DVT-PD is thought of being an international solution. As of 2021, 3.8 billion people own a smart phone out of the possible 7.674 billion people in the world, which is roughly 50%. Further research may be needed to pick out a small speaker for the auditory signal and the vibration mechanism. The speaker and vibration might need a separate circuit and power supply, but this may not be known until the NMES specifications are known. After actuation, DVT-PD may have an appropriate time delay before starting to sense their activity again, so that the patient is not receiving any unnecessary actuations. Once specific hardware and protocols are selected and finalized that meet DVT-PD's goals, this control system may be expanded upon to a more granular level of control (FIG. 5).

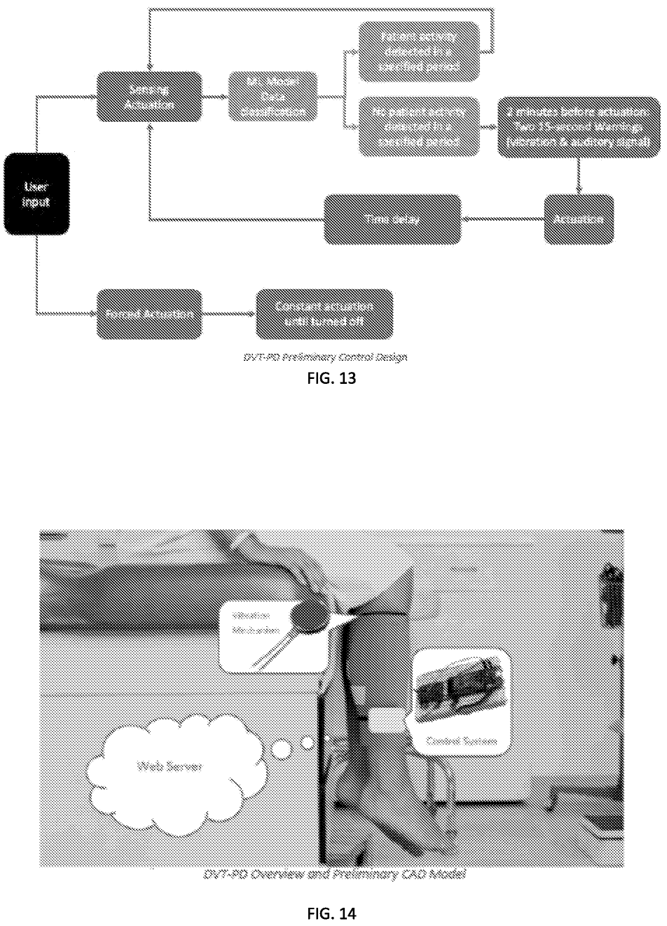

FIG. 13 shows a control design where the user input in FIG. 13 is referring to the patient's operating medical provider (OR medical provider) and their registered medical provider (RN). The OR medical provider is the medical provider who may be helping the surgeon perform the surgery in the operating room and may be responsible for putting DVT-PD on the patients' legs and putting DVT-PD in the forced actuation mode while the patient is sedated. This may provide constant actuation during the patient's most vulnerable period of time. After the surgery, the patient may be taken out of the OR room and to their new recovery room. They may then have a RN who may be monitoring them while they recover. Once the patient is awake from sedation, this medical provider may be responsible for putting DVT-PD in its sensing mode. The RN may then establish the appropriate intensity of stimulation that may be provided to each of the patient's lower legs. She may quickly be increasing the intensity of the signal up until just before the patient's tolerance. This is to assure maximum comfort and compliance for the patient while DVT-PD is actuating.

### 2.2.6 Mechanical Design

The design may resemble a design similar to an ankle brace that is strapped to the lower leg. In this case, it may wrap around the lower extremity and have adjustable slots to allow for different anode and cathode locations that are patient specific; there may be adjustment options for the placements. The device may compensate for pressure distribution and patient comfort. FIG. 14 shows how the device is attached to the user and the respective components integrated into it. The device may be designed to avoid as much pressure as possible to minimize discomfort and avoid DVT risks. As shown, the device may resemble a sleeve and be composed of a material that is electrically insulated, lightweight, and comfortable. The sleeve may cover the leg from the upper calf to the lower leg and may be made in multiple sizes to accommodate patients of various conditions.

A housing may contain the NMES circuit and control system. The housing may be aesthetically pleasing and unobtrusive as there may be phycological issues associated with a box-like cover strapped around the ankle for patients. Housing designs may feature smooth edges, bright colors to help medical providers identify the patients, and possibly a curved design so the housing does not just sit on the side of the lower leg.

### 2.3 Concept Evaluation

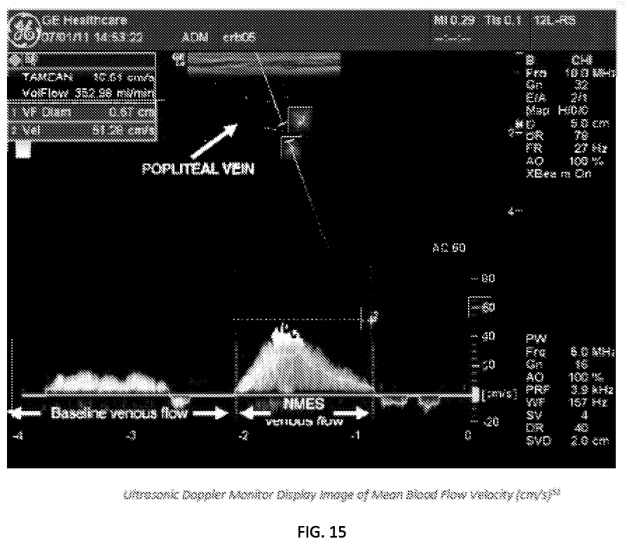

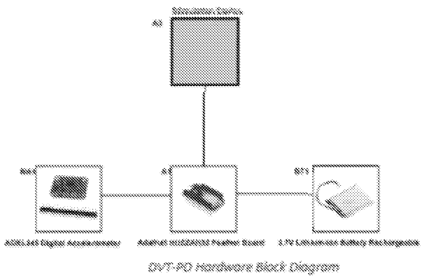

Ultrasonic doppler devices were used to evaluate the DVT-PD effectiveness in use. Ultrasonic doppler devices have been a standard in the medical industry for obtaining images and data inside of the body in a non-invasive fashion. Ultrasonic doppler monitors use reflected sound signals to determine what is going on inside the body. The ultrasound works by first sending sound waves at a very high frequency (typically 1-8 MHz) out from the transducer probe, through some gel that is put on patient's skin. This gel is used to reduce the air space between the transducer and the user's skin in order to reduce the acoustic impedance and reflection that sound waves experience when air is the medium. This gel is important in obtaining a clear and accurate image. After the sound waves travel through the gel and skin, they then come into contact with everything inside the body (veins and blood vessels), and then are reflected back through the skin, gel, and into the transducer. The computer that is a part of the ultrasound doppler monitor then processes, calculates, and outputs the desired image and/or data. The type of ultrasonic device that may be used may ideally have a software and computer that outputs an image of the veins deep in the lower leg, the blood pressure, and most importantly, the mean blood flow velocity as shown down below in FIG. 15.

Since the area of interest is the veins deep inside the tissue of the lower leg, a higher frequency has been shown to be the most effective and accurate, usually ranging from 4-8 MHz (prefer 8 MHz). In order to get a full and accurate depiction of the effectiveness that DVT-PD has at increasing the mean blood flow velocity, the ultrasonic doppler monitor may be collecting data before, during, and after actuation.

### 3 Test Results

### 3.1 Concepts

FIG. 16 shows a block diagram of how DVT-PD may be physically connected. The microcontroller, HUZZAH ESP32 Feather Board, may be the central operational command center of DVT-PD, as it receives position information from the accelerometer, sends data to the server with its built-in WIFI capabilities, analyzes and concludes the need for actuation, then orchestrate actuation if needed.

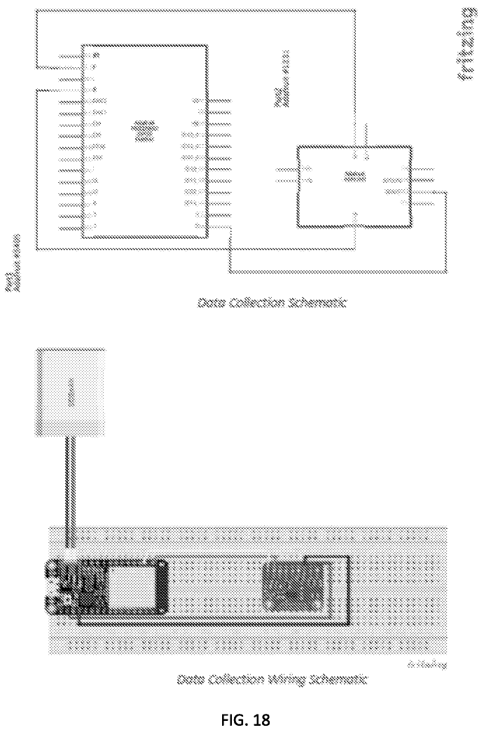

The data collection schematic, as well as the wiring schematic of the data collection component can be seen in FIGS. 17 and 18.

### 3.1.1 Design Implementation

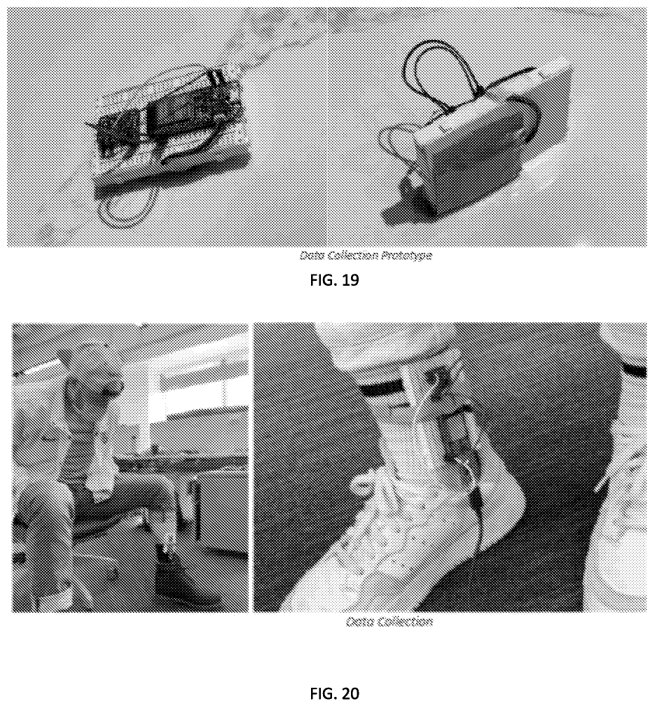

The inventors wired up the microcontroller, the accelerometer, as well as the rechargeable battery according to the schematic in FIG. 19. The inventors chose a smaller breadboard for now to conserve space and is planning on using a solderable breadboard module with housing in the future. Currently, the battery is taped up in the back of the breadboard, but its location is likely to change for the prototype to avoid any potential pressure sores on the patient.

Arduino scripts were created to collect accelerometer data. Once the code was proven to be working correctly, the team tested out the data collection prototype by strapping the prototype to members' legs in various positions to simulate the activities of a patient (FIG. 20).

In addition to building the single layer feedforward neural network, the team also looked into the possibilities of utilizing more complex Deep Learning (DL) models such as convolutional neural network (CNN), long-short term memory (LSTM) neural networks, and convolutional LSTM (ConvLSTM) neural networks. The plan is to add meaningful new features, such as RMS, mean and variance, and running the aforementioned models side by side with the current model (with smaller computational complexity, feature space and computation time) to see which one has the highest accuracy.

### 3.2 Additional Analyses

One challenge to overcome is to achieve a high accuracy of classification with just an accelerometer, instead of having to use both an accelerometer and an EMG, since this may allow for a cheaper and more portable design. A python script was used to generate synthetic data to model the structure of the data with x, y, z coordinates and a specific class corresponding to 10 minutes of data collection with a sampling rate of 200 Hz. A short example of normalized synthetic data is shown in FIG. 30, Table 6. The classes, ranging from 0-2, were selected corresponding to the target market of hospitalized patients, which are sitting/standing, lying down, and walking.

Before feeding this data into the machine learning model, some data preprocessing was performed. Firstly, the data needs to have the same number of data points for each class so that the model does not oversample on a specific class. To do this, a python script was written that takes in all the data in csv form, then automatically equalizes it so there is an equal amount of data per class. Secondly, the data needs to be normalized. Several normalizing techniques were explored such as using z-scores, a Fisher discriminant ratio, and 0-1 normalizing. The last method was chosen for simplicity and all values were divided by the largest amount to be between 0 and 1.

After the ML model deciphers user activity states and determines the need to actuate, the NMES circuit is activated and performs stimulation on the patient. The subsequent section goes over results found from the design process of the NMES circuit.

### 3.3 Actuation

DVT-PD may be actively sensing and classifying the patient's movement and then need to actuate once the patient is classified as being at risk for developing a blood clot or. DVT-PD may also need to be able to adjusts the stimulations intensity from low to high. In order for all of this to be achieved, an NMES device may need to be controlled by the microcontroller (which may be communicating with the server where the ML classifier may be running). Circuit schematics may achieve the desirable voltage and current output safely, while being able to be powered for the desirable time. The key for the NMES circuit is to be able to incrementally raise the intensity between low and high so that the patients can receive the proper intensity of stimulation while still being comfortable. This feature may increase the likelihood that the patient may use DVT-PD.

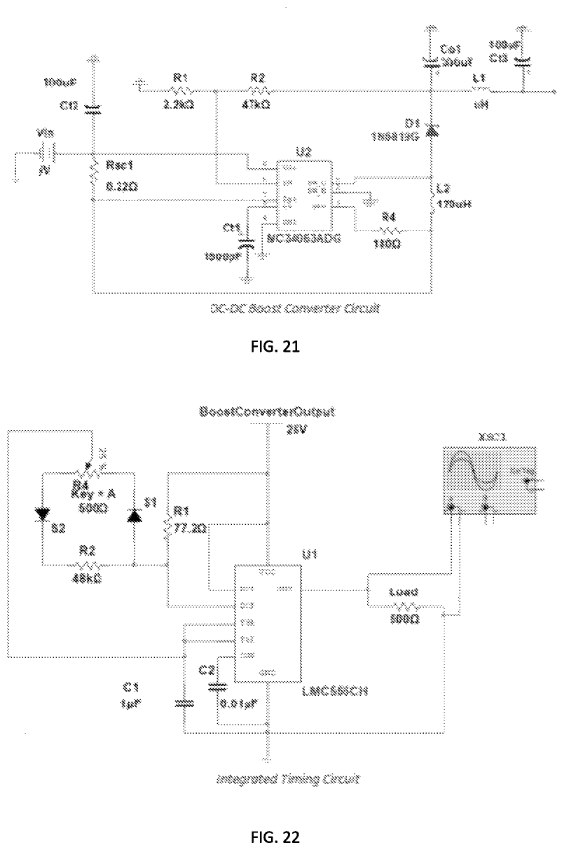

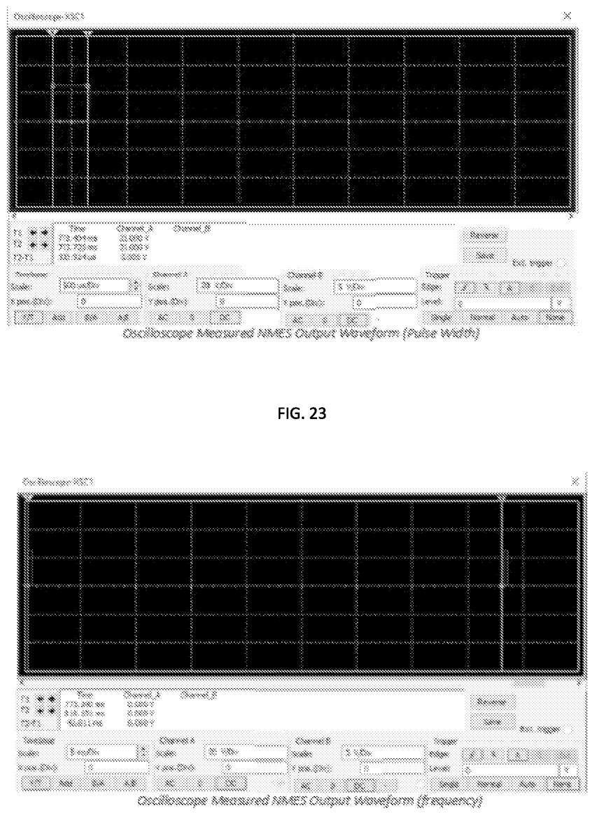

The NMES circuit being described may include a DC-DC boost converter and an integrated circuit timer. The DC-DC boost converter and the integrated circuit timer are shown in FIGS. 21 and 22 respectively. The boost converter is responsible for amplifying the microcontroller's 5V output to the desired voltage, which is applied to the patient's lower leg by the electrodes. The Vin (5V DC) in FIG. 21 models the output of the microcontroller and is boosted up to 20V DC. The output signal of the boost converter passes through a filter that stabilizes the output waveform. The output voltage feeds into the voltage common collector (Vcc) of the timer.

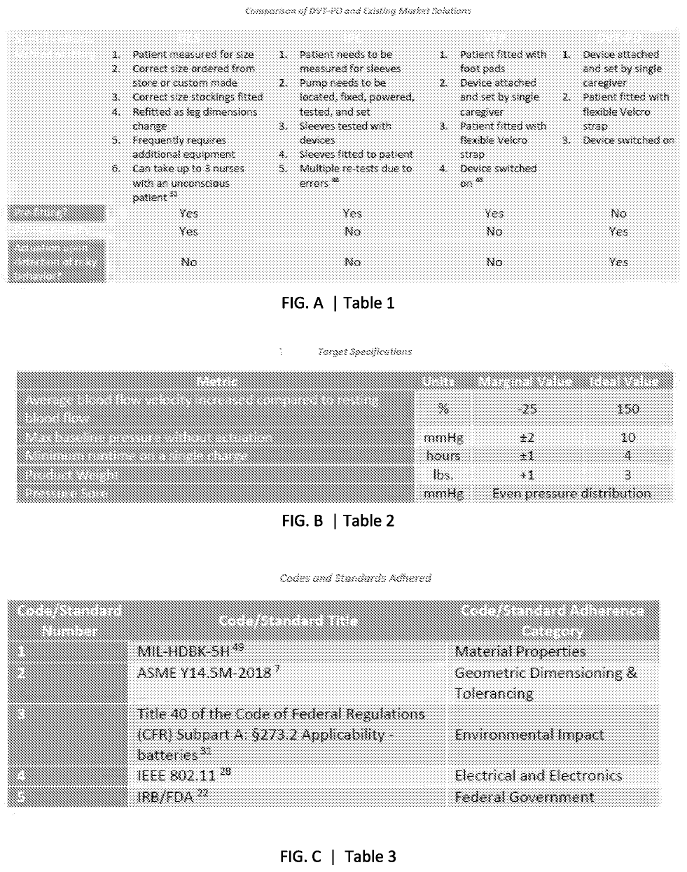

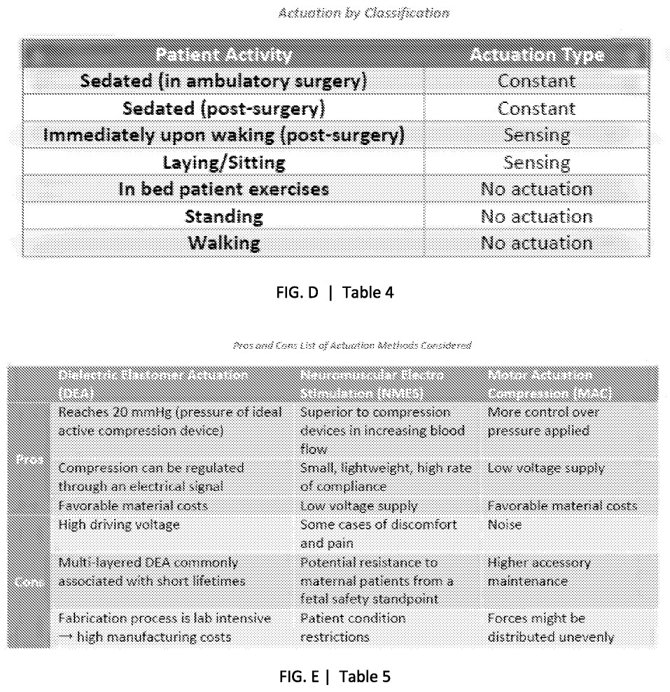

Upon receiving the boost converter's constant 20V DC output, the integrated timer circuit was responsible for regulating the signal's frequency and pulse width to the ranges of 10-50 Hz and 300-400 us, respectively, as shown in FIG. 22. The final output waveform (FIG. 23), measured with an oscilloscope, displayed a pulse width of 320 us and voltage of 20V. The potentiometer (R4) was crucial for adjusting the pulse width of the square pulses without affecting the frequency. The ratio of R1/R2, as well as capacitor C1 determined the desired pulse frequency of 10-50 Hz. The period of the output waveform was 24.811 ms, resulting in a frequency of 23.4 Hz (FIG. 24). The 500 (load modeled the estimated average muscle and fat resistance of the lower leg. The signal across the 500Ω load had a current of 50 mA DC, which was within the desired intensity range for the NMES signal.

### 4 Discussion

After equalizing and normalizing, the aforementioned synthetic data was trained on the machine learning classifier with 10 epochs, and an accuracy of 86.7% was obtained. This result was very promising due to the random nature of the data, and a higher accuracy is expected when an IRB-approved study is conducted with real volunteering subject's data.

It should be noted that while the team envisions many added features and extensions of the device, it is more realistic that the goal for this project is laboratory and initial on-body testing to establish performance.

### 5 Materials/Resources

### 5.1 Hardware

The hardware being used currently mainly may include a Adafruit HUZZAH32 Feather Board (microcontroller), ADXL345 Digital Accelerometer (position sensor), a breadboard, wires and a Lithium-Ion Polymer Battery (power source).

The microcontroller chosen was the HUZZAH32 Feather Board. Affordably priced, this board also has a built-in USB-to-Serial converter, automatic bootloader reset, and a lithium ion/polymer charger. These added built-in features effectively solved some of the problems associated with the ESP8266 HUZZAH board, which was previously considered and tested by the team, and optimized the space of the data collection component of the design.

The ADXL345 Digital Accelerometer is a lower power, 3-axis accelerometer module with both I2C and SPI interfaces. This board is small and features on-board 3.3 voltage regulation and level shifting, which makes it the ideal candidate to interface with 5V microcontrollers such as the HUZZAH feather board. It may include a micro-machined structure on a silicon wafer, which is suspended by polysilicon springs that defects smoothly in any direction when subjected to acceleration in the X, Y and Z directions. The accelerometer provides real-time position and orientation information and is crucial to the project in determining if the accelerometer has changed positions in a period of time—an indication of the activity level of a DVT-PD user.

The table of equipment can be seen in Appendix B, Table 8. Due to COVID-19, separate sets of hardware were ordered so team members could work on this separately.

### 5.2 Software

The software being used may include a CAD software like Fusion 360, Arduino IDE, and a flask web server to collect data and host our machine learning classifier. The CAD software may be used to develop a model of our prototype that includes housing where the control system and NMES circuit may be placed inside. The Arduino IDE is used to control the accelerometer sensing. The flask web server that contains an SQlite database may be used as a destination for the data collected from the accelerometer. The server is also where the processing of the data may occur and may be what controls if an actuation signal is sent.

### 6. Embodiments

Embodiment 1. A deep vein thrombosis prevention device comprises: graduated compression stockings that apply varying amounts of compression to different parts of a user's leg, with greatest pressure at an ankle and lowest pressure at a top of the graduated compression stockings, wherein a pressure gradient encourages that blood to flow upwards towards a user's heart; and a control system that works with a machine learning model to sense the user's dynamic or static movements, and upon receiving data from a sensor in the stockings, the machine learning model classifies, manages, directs, and regulates signals and, together with the control system, directs the stockings to apply the varying amounts of compression.

Embodiment 2. The device of embodiment 1, wherein the device does not immobilize the user.

Embodiment 3. The device of embodiment 1, wherein the device has sensing mechanisms that observe risk within the user, both dynamically and statically.

Embodiment 4. The device of embodiment 1, wherein the device actuates when user risky behavior is identified.

Embodiment 5. The device of embodiment 1, wherein the device has an integrated machine learning model for classifying a user activity state.

Embodiment 6. The device of embodiment 1, wherein the compression increases blood flow velocity in the deep leg veins by 150%.

Embodiment 7. The device of embodiment 1, wherein the sensor uses electromyography.

Embodiment 8. The device of embodiment 7, wherein the sensor uses needles.

Embodiment 9. The device of embodiment 7, wherein the sensors are pads.

Embodiment 10. The device of embodiment 1, wherein the sensor comprises an accelerometer.

Embodiment 11. The device of embodiment 1, wherein the graduated compression stockings comprise an actuator that applies the varying amounts of compression.

Embodiment 12. The device of embodiment 11, wherein the actuator is a dielectric elastomer actuator.

Embodiment 13. The device of embodiment 11, wherein the actuator is a neuromuscular electrical stimulation device.

Embodiment 14. The device of embodiment 11, wherein the actuator is a motor actuated compression device.

Embodiment 15. A deep vein thrombosis prevention device comprising: graduated compression stockings that apply varying amounts of compression to different parts of a user's leg wherein a pressure gradient encourages that blood flows upwards towards the user's heart; and a control system that directs the stockings to apply the varying amounts of compression based on sensor data received.

While the invention has been described with reference to the embodiments herein, a person of ordinary skill in the art may understand that various changes or modifications may be made thereto without departing from the scope of the claims.

1. A deep vein thrombosis prevention device comprising:
graduated compression stockings that apply varying amounts of compression to different parts of a user's leg, with greatest pressure at an ankle and lowest pressure at a top of the graduated compression stockings, wherein a pressure gradient encourages that blood to flow upwards towards a user's heart; and
a control system that works with a machine learning model to sense the user's dynamic or static movements, and upon receiving data from a sensor in the stockings, the machine learning model classifies, manages, directs, and regulates signals and, together with the control system, directs the stockings to apply the varying amounts of compression.

2. The device of claim 1, wherein the device does not immobilize the user.

3. The device of claim 1, wherein the device has sensing mechanisms that observe risk within the user, both dynamically and statically.

4. The device of claim 1, wherein the device actuates when user risky behavior is identified.

5. The device of claim 1, wherein the device has an integrated machine learning model for classifying a user activity state.

6. The device of claim 1, wherein the compression increases blood flow velocity in the deep leg veins by 150%.

7. The device of claim 1, wherein the sensor uses electromyography.

8. The device of claim 7, wherein the sensor uses needles.

9. The device of claim 7, wherein the sensors are pads.

10. The device of claim 1, wherein the sensor comprises an accelerometer.

11. The device of claim 1, wherein the graduated compression stockings comprise an actuator that applies the varying amounts of compression.

12. The device of claim 11, wherein the actuator is a dielectric elastomer actuator.

13. The device of claim 11, wherein the actuator is a neuromuscular electrical stimulation device.

14. The device of claim 11, wherein the actuator is a motor actuated compression device.

15. A deep vein thrombosis prevention device comprising:
graduated compression stockings that apply varying amounts of compression to different parts of a user's leg wherein a pressure gradient encourages that blood flows upwards towards the user's heart; and
a control system that directs the stockings to apply the varying amounts of compression based on sensor data received.
